(19) United States
(12) Patent Application Publication
(10) Pub. No.: US 2007/0093493 A1
(43) Pub. Date: Apr. 26, 2007

(54) Treatment of benign prostatic hypertrophy and lower urinary tract symptoms

(75) Inventors: Bela S. Denes, Kirkland, WA (US); Kenneth M. Ferguson, Seattle, WA (US); John S. Whitaker, Woodinville, WA (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: LILLY ICOS LLC, Wilmington, DE (US)

(21) Appl. No.: 11/545,173

(22) Filed: Oct. 10, 2006

Related U.S. Application Data
(60) Provisional application No. 60/725,772, filed on Oct. 12, 2005.

(51) Int. Cl. A61K 31/498

(52) U.S. Cl. 514/249

(57) ABSTRACT

A method of treating symptoms of benign prostatic hypertrophy and a method of treating lower urinary tract symptoms, are disclosed. The method includes administering to a mammal about 1 to about 20 milligrams of an agent that inhibits cyclic guanosine 3,5-monophosphate specific phosphodiesterase type 5.

### CROSS REFERENCE TO RELATED APPLICATIONS

This application claims the benefit of U.S. Provisional Patent Application Ser. No. 60/725,772, filed Oct. 12, 2005.

### FIELD OF THE INVENTION

The present invention relates to the use of a cyclic guanosine 3′,5′-monophosphate specific phosphodiesterase type 5 (PDE5) inhibitor in a treatment for benign prostatic hypertrophy (BPH) and in a treatment for lower urinary tract symptoms (LUTS).

### BACKGROUND OF THE INVENTION

The prostate gland is a male organ of about chestnut size that surrounds the cervix of the vesical outlet. A benign growth of the prostate gland may result in severe difficulties in micturition up to anuria.

Benign Prostatic Hypertrophy (BPH), also termed Benign Prostatic Hyperplasia, is a chronically progressive, nearly universal, condition in aging men characterized by a nodular enlargement of prostatic tissue resulting in variable degrees of bladder outlet obstruction due to an obstruction of the urethra. BPH is not a major cause of death, but it is a leading cause of morbidity in elderly men and is associated with a variety of lower urinary tract symptoms (LUTS).

LUTS in males include an increased frequency of urination, nocturia, a poor urine stream, and hesitancy or delay in starting the urine flow, for example. Chronic consequences of BPH can include hypertrophy of bladder smooth muscle, a decompensated bladder, bladder stones, renal dysfunction, and an increased incidence of urinary tract infection.

The specific biochemical, histological, and pharmacological properties of the prostate adenoma leading to the bladder outlet obstruction are not yet well understood. However, the development of BPH is considered to be an inescapable condition afflicting the aging male population. BPH is commonly observed in men over the age of 50, and is observed in approximately 70% of the males over the age of 70.

Because no methods are known to prevent or cure BPH, the primary focus of BPH treatment is to alleviate symptoms of BPH and improve the quality of life. Symptoms of BPH include hesitancy (i.e., difficulty in starting to pass urine), weak urine stream, a need to strain to pass urine, a full bladder feeling after urination, an urgent need to pass urine, frequent urination (especially several times at night, nocturia), a burning sensation or pain when urinating, leaking or dribbling urine, and incontinence.

Currently, in the United States, the method of choice for treating BPH is the administration of α-adrenergic blockers and, to a lesser extent, surgery, typically involving transurethral resection of the prostate (TURP). The limitations of surgical treatment of BPH include morbidity associated with an operative procedure in elderly men, persistence or recurrence of obstructive and irritative symptoms, and the significant cost of surgery.

LUTS is recognized as a separate condition which, although traditionally associated with BPH, is now known to have other etiologies as well. LUTS also is recognized as being associated with males and females. LUTS comprises three groups of symptoms, i.e., irritative, obstructive, and postmicturition symptoms. Irritative, or storage, symptoms comprise urgency, frequency, and nocturia. Obstructive, or voiding, symptoms are the identical symptoms associated with BPH.

Although females do not develop morphological BPH, females do suffer from LUTS due to unstable bladder contractions. Such unstable bladder contractions, and LUTS due to unstable bladder contractions, appear with greater frequency in females as the female population ages. Although LUTS in males and females share common features, the underlying pathophysiology may be different. LUTS in women manifests itself primarily in an increased frequency of urination and poor control of voiding.

In the development of BPH, the glandular portions of the prostate gland increase by double their volume, and the muscular and fibrous portions increase by four times their volume. Because these muscle cells account for a large portion of the total prostatic tissue (at least 35%), a distinct improvement of micturition can be achieved by a pharmacologically induced relaxation of these muscle cells. The compounds used to date typically (a) belong to the group of alpha-receptor blockers or (b) interfere with the hormonal regulation of the prostate gland. These therapeutic treatments have been characterized by a low effectiveness, a slow onset of action, significant side effects, or a combination of such disadvantages.

Agents that elevate cGMP levels are well known and can work through any of several mechanisms. In particular, PDE5 inhibitors are widely known as cardiovascular agents for the treatment of conditions such as angina, hypertension, and congestive heart failure, and for the treatment of impotence, importantly by oral administration. U.S. Patent Publication 2003/0199517 discloses use of PDE1, PDE4, and PDE5 inhibitors in the treatment of prostatic diseases.

### SUMMARY OF THE INVENTION

The present invention provides a method of treating BPH, and a method of treating LUTS, comprising administering to a mammal in need of such treatment about 1 mg to about 20 mg of (6R,12aR)-2,3,6,7,12,12a-hexahydro-2-methyl-6-(3,4-methylenedioxyphenyl)pyrazino[2′,1′:6:1]pyrido[3,4-b]indole-1,4-dione (Compound I), preferably in a form suitable for oral administration. Compound (I) has been assigned the general name of “tadalafil.”

The present invention also provides a method of treating BPH in male mammals and LUTS in male and female mammals, including male and female humans, comprising administering to the mammal about 1 mg to about 20 mg of Compound (I) chronically, and preferably daily. The present treatment method reduces the frequency or severity of at least one symptom of BPH or LUTS.

The present invention further provides a method of treating symptoms of BPH, and to a method of treating LUTS, comprising coadministering to a mammal in need thereof about 1 mg to about 20 mg of Compound (I) and a second therapeutic agent capable of treating BPH or LUTS, for example, an α-adrenergic antagonist or an inhibitor of another phosphodiesterase. Compound (I) and the second therapeutic agent can be administered simultaneously, either together in the same composition or separately in discrete dosage units, or in sequence.

The present invention also provides a method of treating symptoms of BPH, and to a method of treating LUTS, and concomitantly treating male erectile dysfunction (MED), by administering to about 1 mg to about 20 mg of Compound (I) to a male mammal that has not previously been diagnosed as suffering from MED. The method is useful in the treatment of males who do not suffer from erectile dysfunction, i.e., are free of erectile dysfunction.

The present invention further provides a kit comprising a first container containing a composition comprising about 1 mg to about 20 mg of Compound (I), an optional second container containing a composition comprising a second therapeutic drug capable of treating BPH or LUTS, and a package insert providing for a chronic, e.g., daily, administration of Compound (I) to treat an individual suffering from BPH or LUTS.

In another embodiment, the first container contains a composition comprising about 1 mg to about 20 mg of Compound (I) and a second therapeutic drug capable of treating BPH or LUTS. In this embodiment, a second container typically is not included in the kit.

### DETAILED DESCRIPTION

For purposes of the present invention disclosed and described herein, the following terms and abbreviations are defined as follows.

The term “container” means any receptacle and closure therefor suitable for storing, shipping, dispensing, and/or handling a pharmaceutical product.

The term “package insert” means information accompanying the product that provides a description of how to administer the product, along with the safety and efficacy data required to allow the physician, pharmacist, and patient to make an informed decision regarding use of the product. The package insert generally is regarded as the “label” for a pharmaceutical product. The package insert incorporated into a present kit indicates that Compound (I) is useful in the treatment of BPH or LUTS.

The term “treatment” includes, but is not limited to, an alleviation, a reduction, a mitigation, a palliation, or a reversal of a progression or severity of a disease, condition, or a symptom of a disease or condition.

The terms “a mammal in need of such treatment” and “in a patient in need thereof” mean a mammal, including humans, exhibiting at least one symptom of BPH or LUTS.

The term “chronic or chronically” refers to the regular administration of Compound (I) in intervals unrelated to the onset of a symptom of BPH or LUTS. To receive the full benefit of the present invention, chronic administration generally refers to regular administration for an extended period, typically daily as long as the patient suffers from BPH or LUTS.

The term “chronic” administration encompasses other regimens in addition to daily dosing. For example, chronic administration encompasses administration of a sustained release formulation that provides sufficient Compound (I) on a regular basis and unrelated to the onset of a symptom of BPH or LUTS. Contrary to an acute or on-demand administration, chronic administration does not link the administration of Compound (I) to the onset of a symptom of BPH or LUTS.

The terms “day” and “daily” refer to the administration of Compound (I) one or more times, generally one to three times, still more preferably one time, per about 24-hour period. “About 24-hour period” refers to a time span of about 20 to about 28 hours.

Compound (I) has the following structural formula:

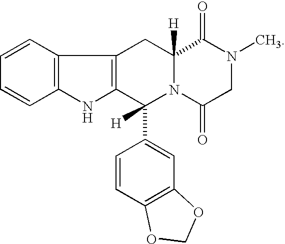

Compound (I) is administered in an amount of about 1 mg to about 20 mg, and provides a clinically significant response in the treatment of BPH and LUTS. The clinical response includes an improvement in the condition treated or in the prevention of the condition.

As noted above, the present invention provides the use of Compound (I) to treat symptoms of BPH and to treat LUTS. For male subjects, Compound (I) can be used to treat males suffering from LUTS, but not BPH, and also can be used to treat males suffering from both LUTS and BPH. In one embodiment, the method comprises orally administering a pharmaceutical formulation comprising Compound (I), daily, to a patient suffering from BPH or LUTS. Compound (I) and its preparation are disclosed in U.S. Pat. No. 5,859,006, incorporated herein by reference.

The present invention is based on clinical experiments and observations that Compound (I), at about 1 mg to about 20 mg, preferably administered daily and using an oral dosage form, effectively treats BPH and LUTS. In accordance with the present invention, Compound (I) can be administered, for example, in dosage amounts of 1, 1.25, 2, 2.5, 3, 4, 5, 7.5, 10, 12.5, 15, 17.5, or 20 mg to effectively treat BPH or LUTS.

The term “oral dosage form” is used in a general sense to reference pharmaceutical products administered orally. Oral dosage forms are recognized by those skilled in the art to include, for example, liquid formulations, tablets, capsules, and gelcaps. In one embodiment, the oral dosage form is a solid dosage form, particularly, tablets comprising about 1 to about 20 mg of Compound (I). Suitable pharmaceutical dosage forms include coprecipitate forms described, for example, in U.S. Pat. No. 5,985,326, incorporated herein by reference.

In preferred embodiments, the unit dosage form of the present invention is a solid free of a coprecipitate form of Compound (I), but rather contains solid Compound (I) as a free drug, for example, as disclosed in U.S. Pat. No. 6,821,975, incorporated herein by reference. The term “free drug” means solid particles of a drug not intimately embedded in a polymeric coprecipitate.

Any pharmaceutically acceptable excipient for oral use is suitable for preparation of such oral dosage forms. Preferably, the oral dosage form comprises pharmaceutical excipients generally recognized as safe such as lactose, microcrystalline cellulose, starch, calcium carbonate, magnesium stearate, stearic acid, talc, and colloidal silicon dioxide, and are prepared by standard pharmaceutical manufacturing techniques as described in Remington's Pharmaceutical Sciences, 18th Ed., Mack Publishing Co., Easton, Pa. (1990). Such techniques include, for example, wet granulation followed by drying, milling, and compression into tablets with or without film coating; dry granulation followed by milling, compression into tablets with or without film coating; dry blending followed by compression into tablets, with or without film coating; molded tablets; wet granulation, dried, and filled into gelatin capsules; dry blend filled into gelatin capsules, or suspension and solution filled into gelatin capsules. Generally, the solid dosage forms have identifying marks which are debossed or imprinted on the surface.

The oral dosage form also can be in the form of a sustained release formulation that chronically provides about 1 to about 20 mg/day of Compound (I) to an individual over the course of a few to several days.

Compound (I) preferably is packaged as an article of manufacture, or kit, for human pharmaceutical use comprising a package insert, a container, and a dosage form comprising about 1 to about 20 mg of Compound (I).

The package insert incorporated into the kit indicates that Compound (I) is useful in the treatment of BPH and LUTS. The package insert also provides instructions to administer one or more about 1 to about 20 mg unit dosage forms, chronically, and preferably daily. Preferably, the dose administered is about 2.5 to about 20 mg/day. Preferred unit dosage forms contain 2.5 mg, 5 mg, 10 mg, or 20 mg of Compound (I).

The container used in the kit is conventional in the pharmaceutical arts. Generally, the container is a blister pack, foil packet, glass, or plastic bottle, and accompanying cap or closure, or other such article suitable for use by the patient or pharmacist. Preferably, the container is sized to accommodate 1 to 1000 solid dosage forms, preferably 1 to 500 solid dosage forms, and most preferably, 5 to 30 solid dosage forms.

Compound (I) can be used in combination with a second therapeutic agent capable of treating BPH or LUTS. The present invention, therefore, encompasses a mixture of Compound (I) and a second therapeutic agent. Such a mixture can be in the form of a composition comprising Compound (I), a second therapeutic agent, and a therapeutically acceptable diluent or carrier. In addition, Compound (I) and a second therapeutic agent can be administered either simultaneously from a single composition or from separate compositions, or sequentially from different compositions. Compound (I) can be administered prior to the second therapeutic agent or vice versa. In addition to the about 1 mg to about 20 mg of Compound (I), the second therapeutic agent is administered in a sufficient amount to provide the desired therapeutic effects with respect to treating BPH or LUTS. The therapeutic effects of Compound (I) and the second therapeutic agent in the treatment of BPH and LUTS can be additive or synergistic.

More particularly, Compound (I) can be coadministered with an α-adrenergic antagonist (also referred to herein as an “α-antagonist”) to treat BPH and/or LUTS. “Coadministration” as used herein, refers to a combination of an α-adrenergic antagonist and Compound (I), means that the individual compounds can be administered together in a composition if the route of administration for each component is the same. “Coadministration” also includes administering Compound (I) and an α-adrenergic antagonist separately, but as part of the same therapeutic treatment program or regimen. It is contemplated that separate administration of each compound, at different times and by different routes, may be recommended. Thus, the two compounds need not necessarily be administered at essentially the same time. It is possible and contemplated that the compounds can be administered at different times, including on different days, but as part of the same regimen. Whether coadministered separately or together in a single composition, it is most preferred that both compounds be administered in an oral dosage form.

The α-antagonist can be selective for either α₁- or α₂-adrenergic receptors (sometimes herein abbreviated as “adrenoceptor”), or it can be nonselective, i.e., exhibiting antagonist activity at both α₁- and γ₂-adrenoceptors. Nonselective antagonists may be used. Antagonists selective for the α₁-adrenoceptor are more preferred.

Useful α-antagonists include, but are not limited to, doxazosin, terazosin, abanoquil, and prazosin, and pharmaceutically acceptable salts thereof, such as doxazosin mesylate, terazosin hydrochloride, abanoquil mesylate, and prazosin hydrochloride, which have been reported to be selective for α₁-adrenoceptors.

Examples of additional α-antagonists include alfuzosin, indoramin, naftopidil, phentolamine, tamsulosin, trazodone, bunazosin, indoramin, dapiprazole, phenoxybenzamine, idazoxan, efaroxan, and yohimbine, and pharmaceutically acceptable salts thereof. Also useful are the rauwolfa alkaloids. Of these, phenoxybenzamine, phentolamine, trazodone, and dapiprazole are reported to be nonselective. Rauwolfa alkaloids, idazoxan, efaroxan, and yohimbine are reported to be selective for α₂ receptors. The other compounds listed above are reported to be selective for α₁ receptors. Further α-antagonists known in the art and reported to be specific for α₁ include: Recordati 15/2739, SNAP 1069, SNAP 5089, RS 17053, and SL 89.0591.

α-Antagonists and salts thereof, in addition to those identified above, are disclosed, for example, in U.S. Pat. Nos. 4,188,390; 4,026,894; 3,511,836; 4,315,007; 3,527,761; 3,997,666; 2,503,059; 4,703,063; 3,381,009; 4,252,721; and 2,599,000, each incorporated herein by reference.

For example, specific α-antagonists for coadministration with Compound (I) include tamsulosin, administered, for example, at about 0.4 to about 0.8 mg per day; alfuzosin, administered, for example, at about 10 mg per day; doxazosin, administered, for example, at about 1 to about 8 mg per day; or terazosin, administered, for example, at about 1 to about 20 mg per day. Exemplary specific doses of the foregoing α-antagonists include: tamsulosin, 0.4 mg once per day or 0.8 mg once per day; doxazosin, 1.0 mg once per day, or 2.0 mg once per day, or 4.0 mg once per day, or 8.0 mg once per day; terazosin, 1.0 mg once per day, or 2.0 mg once per day, or 5.0 mg once per day, or 10.0 mg once per day, or 20.0 mg once per day.

The α-antagonism of a compound can be determined using a number of conventional assays in vitro. Suitable assays include those disclosed in U.S. Pat. No. 5,599,810 and U.S. Pat. No. 5,340,814, each incorporated herein by reference.

Other second therapeutic agents capable of treating BPH or LUTS, and that can be used in combination with Compound (I), are the 5-α-reductase inhibitors, including, but not limited to, dutasteride and finasteride. Phytopharmaceuticals useful in the treatment of BPH or LUTS also are useful in the present invention when coadministered with Compound (I). Such phytopharmaceuticals include, but are not limited to, saw palmetto berry, β-sitosterol, cernilton, and Pygeum africanum (Tadenan).

Other compounds that can be coadministered with Compound (I) include inhibitors of other phosphodiesterases, in particular phosphodiesterases whose activity is associated with relaxation of smooth muscle or other physiological phenomena involved in BPH or LUTS. For example, inhibitors of cyclic AMP-specific phosphodiesterase type 4 (PDE4), or phosphodiesterase type 3 (PDE3) can be employed. PDE3 inhibitors include, for example, cilostamide, cilastazol, enoximone, ibudilast, imazodan, milrinone, quazinone, trequinsin hydrochloride, and zardaverine (a PDE3/4 inhibitor). PDE4 inhibitors include, for example, 4-(3-butoxy-4-methoxybenzyl)imidazolidin-2-yl, etazolate hydrochloride, rolipram, and YM 976. Other PDE4 inhibitors are disclosed in U.S. Pat. Nos. 5,665,754; 6,258,833; 6,294,561; 6,313,156; 6,348,602; 6,362,213; 6,372,777; 6,716,871; 6,376,489; 6,680,336; 6,569,890; 6,569,886; 6,500,856; 6,458,787; 6,455,562; and 6,444,671; each incorporated herein by reference.

The ability of Compound (I) or a coadministration of Compound (I) and a second therapeutic agent to treat symptoms of BPH and to treat LUTS can be demonstrated by in vivo tests known in the art. For example, a rat model can be employed as disclosed in U.S. Pat. No. 5,726,202 (see example 1 therein). An anesthetized dog model may also be employed as disclosed in U.S. Pat. No. 4,755,507. Both patents are incorporated herein by reference.

The routes of administration of Compound (I) administered alone, or with a second therapeutic agent, either separately or together in a composition, can be any of those known to the art such as oral, buccal, nasal, parenteral via by intravenous injection, by injection via subcutaneous or intramuscular depot, or transdermal. Oral administration is preferred.

The present invention is based on experiments and observations that BPH and LUTS can be treated using a chronic, low dose of Compound (I). A chronic, and preferably daily, dosing regimen of about 1 to about 20 mg of a Compound (I) also provides other benefits, including no to low adverse effects attributed to the administered low dose of Compound (I).

The efficacy and safety of Compound (I) administered once a day for LUTS secondary to BPH in male humans was tested. In particular, the efficacy and safety of Compound (I) dosed once a day was assessed in men suffering from moderate to severe LUTS secondary to BPH in a randomized, double-blind, placebo-controlled, parallel-group, dose-escalation study.

Patients were treated with placebo for four weeks (single blind) to evaluate treatment compliance and establish baseline International Prostate Symptom Score (IPSS) and uroflowmetry values. After stratification by baseline IPSS (13-19, moderate LUTS; 20-35, severe LUTS), geographic region, and prior α-blocker therapy, 281 men were randomly assigned to two groups: (1) Compound (I) (5 mg for 6 weeks followed by dose escalation to 20 mg for 6 weeks) or (2) placebo (12 weeks). The primary efficacy end point was change in IPSS at 6 and 12 weeks. Secondary efficacy endpoints included changes in IPSS Quality of Life (QoL) index, BPH Impact Index (BII), uroflowmetry values, and a Global Assessment Question (LUTS GAQ) (Has the treatment you have been taking since your last visit improved your urinary symptoms?). Responses to Compound (I) 5 mg (6 weeks) or Compound (I) 5 mg followed by 20 mg (5/20 mg; 12 weeks total) were compared with placebo using ANCOVA (IPSS, BII, uroflowmetry values) and logistic linear regression (LUTS GAQ) models.

At 6 weeks (5 mg) and 12 weeks (5/20 mg) Compound (I) improved change from baseline scores for IPSS, IPSS QoL, BII, and LUTS GAQ compared with placebo. Peak flow rate (Qₘₐₓ) changes were similar in placebo and Compound (I) treatment groups. The subset of men with LUTS/BPH who were sexually active and also had erectile dysfunction showed a significant increase in International Index of Erectile Function (IIEF) EF domain scores (6.0 in 5 mg vs. 0.6 placebo; 7.7 in 5/20 mg vs. 1.4 placebo; LS Means, n=78, 74) (p<0.001).

In summary, Compound (I) dosed once a day was well tolerated and demonstrated statistically significant and clinically meaningful efficacy in the treatment of LUTS secondary to BPH, and also improved erectile function in men with both LUTS and MED. The onset of improvement in LUTS is within four weeks of treatment initiation.

What is claimed is:

1. A method of treating benign prostatic hypertrophy (BPH) comprising administering to a male mammal in need of such treatment about 1 to about 20 mg of a Compound (I) having a structure

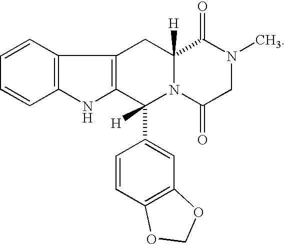

2. The method of claim 1 wherein the treatment reduces the frequency or severity of at least one symptom of BPH.

3. The method of claim 2 wherein the symptom is selected from the group consisting of hesitancy, weak urine stream, full bladder feeling, frequent urination, nocturia, burning sensation or pain during urination, leaking or dribbling of urine, and incontinence.

4. The method of claim 1 wherein Compound (I) is administered chronically.

5. The method of claim 4 wherein Compound (I) is administered daily.

6. The method of claim 1 wherein the Compound (I) is administered in an amount of about 2 to about 20 mg.

7. The method of claim 1 wherein the Compound (I) is administered in an amount of about 2.5 to about 20 mg.

8. The method of claim 1 wherein the Compound (I) is administered in an amount of about 5 to about 20 mg.

9. The method of claim 1 wherein the Compound (I) is administered in an amount of about 2.5 mg.

10. The method of claim 1 wherein the Compound (I) is administered in an amount of about 5 mg.

11. The method of claim 1 wherein the Compound (I) is administered in an amount of about 10 mg.

12. The method of claim 1 wherein the Compound (I) is administered in an amount of about 15 mg.

13. The method of claim 1 wherein the Compound (I) is administered in an amount of about 20 mg.

14. The method of claim 1 wherein the Compound (I) is administered orally.

15. The method of claim 15 further comprising administering a second therapeutic agent capable of treating BPH.

16. The method of claim 15 wherein the second therapeutic agent is selected from the group consisting of an α-adrenergic antagonist, a 5-α-reductase inhibitor, a phytopharmaceutical, a phosphodiesterase inhibitor capable of treating BPH, and mixtures thereof.

17. The method of claim 15 wherein the second therapeutic agent comprises tamsulosin and is administered in an amount of about 0.4 to about 0.8 mg per day.

18. The method of claim 15 wherein the second therapeutic agent comprises alfuzosin and is administered in an amount of about 10 mg per day.

19. The method of claim 15 wherein the second therapeutic agent comprises doxazosin and is administered in an amount of about 1 to about 8 mg per day.

20. The method of claim 15 wherein the second therapeutic agent comprises terazosin and is administered in an amount of about 1 to about 20 mg per day.

21. The method of claim 15 wherein the second therapeutic agent is administered simultaneously with the Compound (I).

22. The method of claim 15 wherein the second therapeutic agent is administered prior to the Compound (I).

23. The method of claim 15 wherein the second therapeutic agent is administered after the Compound (I).

24. The method of claim 1 wherein the male mammal is free of erectile dysfunction.

25. The method of claim 1 wherein, compared to a placebo, an improvement of at least 1.5 points form a placebo-adjusted baseline, as measured by the International Prostate System Score, is achieved after 12 weeks of treatment using 5 mg of Compound (I) per day.

26. The method of claim 1 wherein a statistically significant improvement in a placebo-adjusted BPH impact index is achieved after 12 weeks of treatment using 5 mg of Compound (I) per day.

27. The method of claim 1 wherein the male mammal is a human male.

28. A method of treating lower urinary tract symptoms (LUTS) comprising administering to a mammal in need of such treatment about 1 to about 20 mg of a Compound (I) having a structure

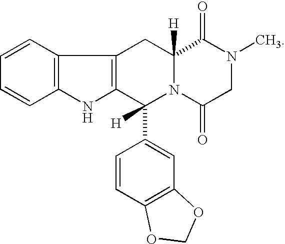

29. The method of claim 28 wherein the symptom is selected from the group consisting of hesitancy, weak urine stream, full bladder feeling, frequent urination, nocturia, burning sensation or pain during urination, leaking or dribbling of urine, and incontinence.

30. The method of claim 28 wherein Compound (I) is administered chronically.

31. The method of claim 28 wherein Compound (I) is administered daily.

32. The method of claim 28 wherein the mammal is a male human.

33. A method of claim 28 wherein the mammal is a female human.

34. The method of claim 28 wherein the compound (I) is administered in an amount of about 2 to about 20 mg.

35. The method of claim 28 wherein the Compound (I) is administered in an amount of about 2.5 to about 20 mg.

36. The method of claim 28 wherein the compound (I) is administered in an amount of about 5 to about 20 mg.

37. The method of claim 28 wherein the compound (I) is administered in an amount of about 2.5 mg.

38. The method of claim 28 wherein the compound (I) is administered in an amount of about 5 mg.

39. The method of claim 28 wherein the compound (I) is administered in an amount of about 10 mg.

40. The method of claim 28 wherein the compound (I) is administered in an amount of about 15 mg.

41. The method of claim 28 wherein the compound (I) is administered in an amount of about 20 mg.

42. The method of claim 28 wherein the Compound (I) is administered orally.

43. The method of claim 28 further comprising administering a second therapeutic agent capable of treating LUTS.

44. The method of claim 43 wherein the second therapeutic agent is selected from the group consisting of an α-adrenergic antagonist, a 5-α-reductase inhibitor, a phytopharmaceutical, a phosphodiesterase capable of treating LUTS, and mixtures thereof.

45. The method of claim 44 wherein the second therapeutic agent comprises tamsulosin and is administered in an amount of about 0.4 to about 0.8 mg per day.

46. The method of claim 44 wherein the second therapeutic agent comprises alfuzosin and is administered in an amount of about 10 mg per day.

47. The method of claim 44 wherein the second therapeutic agent comprises doxazosin and is administered in an amount of about 1 to about 8 mg per day.

48. The method of claim 44 wherein the second therapeutic agent comprises terazosin and is administered in an amount of about 1 to about 20 mg per day.

49. The method of claim 28 wherein the second therapeutic agent is administered simultaneously with the Compound (I).

50. The method of claim 28 wherein the second therapeutic agent is administered prior to the Compound (I).

51. The method of claim 28 wherein the second therapeutic agent is administered after the Compound (I).

52. The method of claim 28 wherein the male mammal is free of erectile dysfunction.

53. A kit for human pharmaceutical use comprising:
(a) an oral dosage form comprising about 1 to about 20 mg of Compound (I);
(b) a package insert providing that Compound (I) is useful to treat benign prostate hypertrophy or to treat lower urinary tract symptoms in a patient in need thereof by utilizing a chronic dosing regimen; and
(c) a container.

54. The kit of claim 53 wherein the chronic dosing regimen is a daily dosing regimen.
